(19) United States
(12) Patent Application Publication
(10) Pub. No.: US 2022/0395437 A1
(43) Pub. Date: Dec. 15, 2022

(54) COMPOSITION FOR THE MAKING OF AN EMULSIFIABLE TRIPHASIC FORMULATION, AND KIT FOR THE USE OF SUCH COMPOSITION

(71) Applicant: Francesco LEVA, TORINO (IT)

(72) Inventor: Francesco LEVA, TORINO (IT)

(21) Appl. No.: 17/755,824

(22) PCT Filed: Nov. 8, 2020
(86) PCT No.: PCT/IB2020/060501

§ 371 Date: May 9, 2022

(30) Foreign Application Priority Data
Nov. 7, 2019 (IT) 102019000020526

(51) Int. Cl. A61K 8/04; A61K 8/73; A61K 8/44; A61K 8/36; A61K 8/365; A61K 8/67; A61K 8/92; A61K 8/9789; A61K 8/64; A61Q 19/00; A61Q 19/02

(52) U.S. Cl. CPC A61K 8/046; A61K 8/735; A61K 8/44; A61K 8/36; A61K 8/365; A61K 8/678; A61K 8/922; A61K 8/9789; A61K 8/64; A61K 8/676; A61K 8/673; A61Q 19/007; A61Q 19/02; A61K 2800/882

(57) ABSTRACT

The invention relates to a composition for the making of an emulsifiable triphasic formulation, which consists of a liquid biphasic composition and a powder solid phase, wherein said liquid biphasic composition consists of a hydrophilic phase and a lipophilic phase. The triphasic emulsifiable composition obtained from the combination of the biphasic liquid composition and the solid powder phase by a kit of the invention is particularly suitable for the use in the clinical-dermatological and/or cosmetological fields.

The present invention refers to a composition and a kit for making of triphasic emulsifiable formulations for topical use, and particularly suitable for the use in the clinical and/or cosmetical field.

### STATE OF THE ART

The skin is a complex organ both for structural and functional organization and it is subject to a continuous renewal which usually takes place every 20-30 days. This process becomes progressively slower with the aging of the cells which have a fundamental role in producing the essential substances of the skin, namely fibroblasts, and lose their functions and are no longer able to produce sufficient quantities of molecules such as the hyaluronic acid, essential to ensure adequate hydration, and amino acids, useful for forming molecules such as collagen and elastin. By allowing the fibroblasts to return to normal functioning by stimulating these cells directly (so-called biostimulation), or providing them with pre-formed substances (so-called bio-restructuring or biorevitalization), means counteracting the signs of aging as well as preventing the onset of a wide spectrum of skin diseases, including skin alterations related to aging, such as keratosis, inflammatory dermatoses, such as rosacea, erythema or eczema, skin diseases due to hormonal alterations, such as acne and hyperpigmentation, and those caused by oxidative stress as well as neoplasms.

More specifically, the term “biostimulation” usually refers to the stimulation of the anabolic functions of skin fibroblasts, i.e. the replication thereof, the synthesis and production of proteins and components of the extracellular matrix (ECM). Aesthetic medicine uses injection and non-injection techniques in order to determine a biostimulation and/or biorevitalization effect, and in order to obtain an improvement in the skin's appearance as well as the barrier and protection, coating and thermoregulation functions thereof.

Polydeoxyribonucleotides, glucosamine sulfate, amino acids and N-acetylcysteine are already known among the biostimulating factors.

The applications for the “bio-restructuring” or “biorevitalization” instead employ different medical devices in order to provide direct supplementation of hyaluronic acid to the skin, alone or linked to other molecules such as amino acids or vitamins. The binding to specific receptors present on the fibroblast surface induces anabolic metabolic effects, allowing a rapid activation of cellular functions in the release zone, without having to synthesize such factors ex novo.

When both bio-stimulating and bio-restructuring methods are applied it is referred to as bio-complementation, which is a process that further amplifies the effect of cellular stimulation with a greater production of structural and enzymatic components, and which induces an increase in cell replication, protein synthesis, and production of the components of the cellular matrix, including glycoproteins, proteoglycans, hyaluronic acid, collagen, elastin, fibronectin, laminin, entactin and various growth factors.

Both bio-stimulating and bio-restructuring or bio-revitalizing have beneficial effects on the skin as, in addition to counteracting spontaneous skin aging (chronoaging) and photo-induced (photoaging), and they determine an optimal condition for the dermal-epidermal functions to work at optimal regimes, prevailing against oxidative damage. The duration of the treatment benefits varies according to the severity of the initial situation and it is related to the lifestyle. In any case, the treatments must be cyclical, i.e. repeated over time and supported by the repetition of the sessions.

There are many types of bio-revitalizing and bio-restructuring products that have found wide use not only in the purely cosmetic sector but also in the clinical field, particularly in the dermatological field, with a curative and preventive function, for example for the treatment of acne and scarring.

However, the therapies that use these products are often based on their administration by subcutaneous or intradermal injection, which is a method characterized by varying degrees of invasiveness and which in any case requires execution by specialized health personnel.

Among the alternatives to said skin bio-stimulating by injection, there are the bio-revitalizing products with a peeling effect, that after topical application they are capable of determining a controlled destruction of the skin layers, with a subsequent dermal and/or dermo-epidermal regeneration. The compositions containing trichloroacetic acid (TCA) have wide value as peeling treatments. They can consist of a single-phase basis or comprise an aqueous phase in combination with an oily phase, i.e. being in a biphasic form. Despite having a modulating exfoliating capacity, TCA-based preparations for local use still require due caution in their use as they are potentially aggressive for the skin. In addition to the potential aggressiveness, they are often characterized by a significant instability, especially at temperatures above 25-30° C., with the risk of a possible breakage by explosion of the containers in which the compositions have been stored.

Therefore, the present invention aims to provide a formulation of a composition having a bio-stimulating and bio-restructuring activity, suitable for topical application, and which overcomes the disadvantages and limitations of the prior art, thus allowing the effective, safe, and simple use thereof in both the clinical and cosmetological field.

### BRIEF DESCRIPTION OF THE INVENTION

The above objects are achieved by the present invention which provides a composition for the realization of an emulsifiable triphasic formulation comprising a synergistic combination of active substances, obtainable from natural sources or by organic synthesis, and which is characterized by a remarkable stability and being particularly effective in the treatment of a wide range of skin alterations.

The present invention also provides a kit for the making of an emulsifiable triphasic formulation according to the appended claims.

Further features and advantages of the invention will be illustrated in detail in the following description and identified in the appended claims.

### DETAILED DESCRIPTION OF THE INVENTION

A detailed description of some preferred embodiments of the composition for the making of a triphasic formulation according to the present invention will now be provided, and for illustrative and non-limiting purposes.

According to the present invention, a biphasic liquid composition and a solid powder phase there are provided. The biphasic liquid composition in turn consists of an hydrophilic phase, preferably aqueous, and of a lipophilic phase.

According to the invention, the biphasic composition and the solid powder phase are physically separated and intended to be extemporaneously mixed, and in order to obtain a triphasic emulsion, particularly suitable for the topical application.

The hydrophilic phase of the invention it is characterized by comprising at least one organic acid and at least one polar amino acid.

Among Organic acids which are suitable for the use in the hydrophilic phase of the composition of the present invention there are, for example, pyruvic acid, mandelic acid, lactic acid, glycolic acid, salicylic acid, ferulic acid, citric acid, malic acid, azelaic acid, kojic acid, and any combination thereof.

Among these, glycolic acid, lactic acid and mandelic acid belong to the class of alpha-hydroxy acids, characterized by having a hydroxy group on the carbon atom adjacent to the carboxyl group. The aforementioned chemical structure allows the alpha hydroxy acid molecules to interlayer between the protein junctions of the corneocytes (small flattened cells devoid of nuclei) of the corneum layer of the epidermis, reducing their cohesion and promoting easier and faster absorption. Following to the action of alpha-hydroxy acids, a thinning of the corneum layer of the skin is obtained, resulting in faster exfoliation of surface dead cells, and greater production of collagen and elastin.

Pyruvic acid is an alpha-ketoacid belonging to the family of fruit acids. Thanks to its low pH, it is particularly suitable for the use thereof in dermatology as an exfoliating substance, in particular for its ability to eliminate pigmented corneocytes with a lightening effect, increasing skin brightness.

Kojic acid is produced by some species of fungi, and is known for a marked depigmenting effect on the skin, probably caused by a specific inhibition that this acid exerts on the tyrosinase enzyme.

In a preferred embodiment, the at least one organic acid is present in the hydrophilic phase of the invention in a total amount comprised between 0.1% and 80%, preferably between 1% and 70%, in weight with respect of the total weight of the hydrophilic phase.

The hydrophilic phase of the composition according to the invention may also comprise azelaic acid and/or salicylic acid.

Both azelaic acid (produced by a “Malassezia furfur” yeast normally found both in the skin flora and in wheat, rye or barley), and salicylic acid have recognized bactericidal properties. They are particularly effective against all types of skin acne.

In a preferred embodiment of the kit of the invention, azelaic acid it is preferably contained in the hydrophilic phase in an amount comprised less than or equal to 10% by weight with respect to the total weight of the hydrophilic phase, and/or salicylic acid it is preferably contained in the hydrophilic phase in an amount less than or equal to 10% by weight with respect to the total weight of the hydrophilic phase.

According to the invention, the hydrophilic phase in the composition of the present invention includes at least one polar amino acid, preferably basic polar amino acid arginine.

In a preferred embodiment, the at least one polar amino acid it is comprised in the hydrophilic phase of the composition according to the invention in the amount comprised between 5% and 50%, and more preferably between 10% and 40% by weight with respect to the total weight of the hydrophilic phase.

In a preferred embodiment of the composition of the invention, the hydrophilic phase comprises the following substances in weight percentage as here below indicated with respect to the total weight of said hydrophilic phase:
pyruvic acid in an amount comprised between 25% and 50% by weight;
mandelic acid in an amount comprised between 5% and 10% by weight;
lactic acid in an amount comprised between 1% and 10% by weight;
glycolic acid in an amount comprised between 1% and 5% by weight;
kojic acid in an amount comprised between 0.1% and 5% by weight;
arginine in an amount comprised between 5% to 50% by weight;
azelaic acid in an amount less than or equal to 5% by weight;
salicylic acid in an amount less than or equal to 5% by weight; and
water in an amount comprised between 20% and 50% by weight.

In the biphasic composition of the present invention, the lipophilic phase is characterized by comprising at least one fat-soluble vitamin, at least one terpene compound, and at least one essential oil.

Preferably, the at least one fat-soluble vitamin is tocopherol. Tocopherol (vitamin E), is a group of fat-soluble vitamins (alpha, beta, gamma and delta-tocopherol and alpha, beta, gamma and delta-tocotrienol). Vitamin E is comprised in many vegetables, for example in fruit, hemp oil, olive oil and especially in wheat germ oil. The main role of vitamin E is to protect the body's tissues from peroxidation reactions and free radicals.

In a preferred embodiment, the at least one fat-soluble vitamin is contained in the lipophilic phase of the composition according to the invention in an amount comprised between 50% and 89%, and more preferably between 50% and 75% in weight with respect to the total weight of the lipophilic phase.

Preferably, the at least one terpene compound contained in the lipophilic phase of the biphasic composition of the invention is bisabolol. This natural alcohol, which can be obtained by distillation of essential chamomile oil, has an effective soothing action on the skin as well as depigmenting, healing and anti-inflammatory activities.

In a preferred embodiment, the at least one terpene compound is contained in the lipophilic phase of the composition of the invention in an amount comprised between 4% and 25%, and more preferably between 10% and 20% by weight with respect to the total weight of the lipophilic phase.

According to the invention, the essential oils contained in the lipophilic phase of the composition of the invention can be of animal or vegetable origin. Among the essential oils suitable for the use thereof in the lipophilic phase of the composition of the invention, olive oil (Olea europaea) and essential oil of bergamot (Citrus bergamia) are here indicated by way of a no limiting example.

In a preferred embodiment, the at least one essential oil is contained in the lipophilic phase of the composition according to the invention in an amount comprised between 1% and 30%, and more preferably comprised between 5% and 25% by weight with respect to the total weight of the lipophilic phase.

In a preferred form of the composition of the invention, the lipophilic phase comprises the following components in a weight percentage with respect to the total weight of said lipophilic phase:
tocopherol in an amount comprised between 50% and 89% by weight;
bisabolol in an amount comprised between 4% and 25% by weight;
olive oil in an amount comprised between 6% and 25% by weight; and
Bergamot essential oil in an amount comprised between 1% and 5% by weight.

The solid powder phase of the composition according to the invention is characterized by comprising hyaluronic acid and/or its salt, ferulic acid, creatine, at least one vitamin and at least one amino acid and/or one of the salts thereof.

Hyaluronic acid (HA) is a polyanionic polymer of natural origin. It is a large, negatively charged linear polymer composed of the repetition of disaccharide units of D-glucuronic acid and N-acetyl-D-glucosamine, joined together alternately by β-1,4 and β-1,3 glycosidic bonds. Hyaluronic acid is the main component of the extracellular matrix (ECM), in particular of the dermal matrix, and belongs to the family of glycosaminoglycans (GAG) and is a molecule which may vary its size.

This molecule has hygroscopic capacity and is responsible for the skin firmness. The integration of the physiological production of this substance, the concentration of which decreases with advancing age, has the beneficial effect of achieving a strengthening of the skin structure, improving its elasticity and filling deep wrinkles from the inside.

In a preferred embodiment, the hyaluronic acid and/or a salt thereof is comprised in the solid powder phase of the composition according to the invention in a quantity comprised between 5% and 10%, preferably between 6% and 9%, by weight with respect to the total weight of the solid phase in powder form.

The hyaluronic acid salt is preferably sodium hyaluronate.

The at least one vitamin comprised in the solid powder phase according to the invention is preferably chosen between ascorbic acid (vitamin C), and riboflavin (vitamin B2).

Among the beneficial effects of the topical use of ascorbic acid there are the antioxidant action which prevent and/or counteract the damage caused by free radicals, the stimulation of collagen production, the reduction of inflammation and irritation as well as the decrease of the pigmentation of skin spots.

In a preferred embodiment, the at least one vitamin comprised in the solid powder phase of the composition of the invention is comprised in an amount between 0.1% and 90%, preferably between 5% and 75% by weight with respect to the total weight of the solid phase in powder form.

Preferably, in the solid powder phase according to the invention, the at least one amino acid and/or its salt is selected from arginine, glycine, proline, hydroxyproline, valine, cysteine hydrochloride, lysine hydrochloride, and any combination thereof. Among these, the amino acids glycine, proline and lysine, precursors of the collagen molecule, are known to have a marked bio-restructuring activity.

In a preferred embodiment, the at least one amino acid and/or a salt thereof is comprised in the solid powder phase of the composition of the invention in a total amount comprised between 0.1% and 7%, preferably between 1% and 5%, by weight of the total weight of the solid powder phase.

According to the invention, ferulic acid and creatine are also comprised in the solid powder phase of the composition.

Among the various activities that ferulic acid shows, of particular interest are its antimicrobial properties, which it performs even at low concentrations.

In a preferred embodiment of the invention, ferulic acid is comprised in the solid powder phase of the composition in an amount comprised between 1% and 5% by weight with respect to the total weight of the solid powder phase.

In another preferred embodiment of the invention, creatine is comprised in the solid powder phase of the composition in an amount comprised between 0.1% and 1% by weight with respect to the total weight of the solid powder phase.

According to the invention, the solid phase in powder can also comprise a synthetic hexapeptide, preferably a palmitoyl hexapeptide, more preferably palmitoyl hexapeptide-19.

Various studies have shown that hexapeptides, and in particular palmitoyl hexapeptide-19, exert a destabilizing action against the process of skin tightening and muscle contractions that generate the appearance of wrinkles on the face. This evidence suggests an activity of hexapeptides substantially similar to that of botulinum toxin, but without the dangerous side effects of the cosmetic and therapeutic use of this protein.

In a preferred embodiment of the invention, the synthetic hexapeptide is preferably comprised in the solid powder phase of the composition in an amount less than or equal to 10% by weight with respect to the total weight of the solid powder phase.

According to another preferred embodiment of the invention, dextran-hydroxypropyltrimony chloride (CAS number 83855-79-2) is also comprised in the solid powder phase of the present composition.

Preferably, the dextran-hydroxypropyltrimony chloride is comprised in the solid powder phase of the composition of the invention in an amount less than or equal to 10% by weight with respect to the total weight of the solid powder phase.

In a preferred form of the composition of the invention, the solid powder phase comprises the following components in weight percentage with respect to the total weight of said solid powder phase:
ascorbic acid in an amount between 75% and 89% by weight;
hyaluronic acid and/or a salt thereof in a quantity comprised between 5% and 10% by weight;
ferulic acid in an amount between 1% and 5% by weight;
creatine in an amount between 0.1% and 1% by weight;
arginine in an amount between 0.1% and 1% by weight;
cysteine hydrochloride in an amount between 0.1% and 1% by weight;
glycine in an amount between 0.1% and 1% by weight;
hydroxyproline in an amount of between 0.1% and 1% by weight;
lysine hydrochloride in an amount between 0.1% and 1% by weight;
proline in an amount between 0.1% and 1% by weight;
valine in an amount between 0.1% and 1% by weight;
riboflavin in an amount between 0.1% and 1% by weight;
dextran-hydroxypropyltrimony chloride (CAS number 83855-79-2) in an amount less than or equal to 0.1% by weight;
palmitoyl hexapeptide-19 in an amount less than or equal to 10% by weight.

Optionally, the solid powder phase of the composition of the invention can also comprise solid inert elements, for example silica microparticles or anti-caking agents in general.

Furthermore, according to the present invention, a kit is provided which provides a device configured to keep the liquid biphasic composition and the solid powder phase separated one from each other until the moment of their blending to become a triphasic emulsion.

For example, the device of the kit according to the invention can be a bottle equipped with a cap having a receiving central cavity bounded by a lower inner perforable wall, for example by a wall of aluminum material. In a device with this configuration, the bottle contains inside thereof the biphasic liquid composition of the invention while the solid powder phase composition of the invention is enclosed in the cap receiving cavity. By piercing the lower wall of the cap receiving cavity, for example with the application of a manual pressure of appropriate intensity, a flow of the solid powder phase contained in the receiving cavity is allowed inside the bottle and where the hydrophilic phase and the lipophilic phase are contained, thus obtaining the combination of the liquid biphasic composition and the solid powder phase composition into a triphasic formulation.

Obviously, the choice of the most suitable device for the packaging of the kit according to the invention is made according to the desired use and is largely within the skills of the average skilled man in the field.

As will be illustrated in more detail in the experimental part which follows, the triphasic emulsifiable formulation above indicated and obtained by combining the biphasic liquid composition and the solid powder phase composition of the kit which is an object of the invention has proved to be endowed with a significant skin bio-stimulating and bio-restructuring activity, with enormous benefits in terms of photo-rejuvenation as well as a considerable chemical-physical stability.

More specifically, the kit according to the invention is particularly suitable for the use in those combined bio-stimulating and bio-restructuring methods known as bio-complementation, wherein the high amount of both direct stimulus components such as the hyaluronic acid, and indirect stimulus such as amino acids, vitamins, alpha and beta hydroxy acids induce an optimal and stimulating environment for the fibroblast. This involves a restoration of normal dermo-epidermal functions and such as to determine an immediate effect on vascularization, trophism, skin tone, elasticity, silkiness and complexion.

The bio-stimulating and bio-restructuring capacity of the present formulation depends on the following synergistic actions of the components of the hydrophilic, lipophilic and solid powder phases:

(i) the essential oils comprised in the lipophilic phase, in particular the purified bergamot essence and extra virgin olive oil, exert a powerful antioxidant function, also supported by a notable content of vitamin E, which has the effect of counteracting the oxidative stress and the damage induced by the latter, especially by blocking the activity of metalloproteinases;

(ii) lipid molecules, being constituents of the barrier that controls water loss, increase the dermal-epidermal retention capacity of the water itself by increasing its hydration, and modulate the entry of alpha and beta hydroxy acids comprised in the hydrophilic phase;

(iii) the alpha and beta hydroxy acids of the hydrophilic phase, in addition to show a sebum-regulating action, they act with an antimicrobial action, for example against pathogenic skin microorganisms such as Propiniobacterium acnes and Staphyilococcus epidermidis, both of which are involved in the pathology of acne. In addition, these hydroxy acids operate a control action on the production of melanin and on the regeneration of the epidermal layers, and consequently on the renewal of the epidermal barrier;

(iv) the synthetic hexapeptide, amino acids and vitamin C, comprised in the solid powder phase, act as stimulants for the regeneration of the constituents of the dermal matrix, such as collagen, elastin, glycoproteins and glycosaminoglycans, restoring an optimal environment similar to young skin, as well as promoting the activity of fibroblasts which, when stimulated, return to an optimal structural and functional situation. In particular, amino acids represent the precursors for the regeneration of the reticular collagen component (type III collagen), glycine, proline and hydroxyproline reduce the acidity of the matrix avoiding the acidification processes and the formation of fibrotic collagen. The presence of the hexapeptide in the solid powder phase also has the effect of inducing a relaxation of the muscle fibrils in the areas of greatest movement, decreasing skin wrinkling due to expression lines;

(v) hyaluronic acid, by acting on the CD44 receptors of fibroblasts, increases the skin's regenerative response;

(vi) the inhibitory effect on tyrosinase and enzymes involved in melanogenesis operated by kojic, azelaic and vitamin C acids counteracts the presence of pathological situations such as chloasma, melasma and post-sclerotherapy hyperpigmentation.

The skin renewal and hydration resulting from the application of the triphasic formulation obtained from the combination of the biphasic liquid composition and the solid powder phase of the kit of the invention, in addition to counteracting ichthyosis and skin dryness typical of aging skin, counteracts the of keratosis and neoformations due to an imbalance in the dermo-epidermal metabolic processes.

With the advantageous combination of the aforementioned features, the kit according to the invention is particularly suitable for the use in the clinical setting, particularly in the dermatological field. With reference to skin diseases may be listed for example but not exclusively, acne pathology, alterations in melanogenesis such as chloasma, melasma, solar or senile hyperpigmentation, skin xerosis, post-sclerotherapy hyperpigmentation, and onychomycosis.

Alternatively, the kit according to the invention may be directed also to the purely cosmetological field, in order to modify or resolve unsightly aspects of the skin caused solely by physiological processes in individuals not affected by dermatological pathologies, for example in individuals who are not affected by acne pathology, alterations of melanogenesis, such as chloasma, melasma, solar or senile hyperpigmentation, and cutaneous xerosis, postclerotherapy hyperpigmentation and from onychomycosis.

The formulation obtained from the combination of both the liquid biphasic composition and the solid powder phase of the kit of the invention is preferably suitable for topical administration, preferably in the form of ointment or spray.

### EXAMPLES

The following examples are provided for illustrative and non-limiting purposes of the scope of the invention as defined in the appended claims.

1. Stability of the Invention Formulation

The inventor have carried out different tests in order to verify the stability of the present formulation when contained in the kit according to the present invention, as well as of the emulsified formulation obtained by blending the liquid biphasic composition and the solid powder phase. In particular, stability and compatibility tests were carried out under different conditions over time.

Thereafter, stability and compatibility tests were carried out on the kit and on the formulation of the invention at temperatures of 4° C., 25° C., 42° C., in no light condition, after exposure to ambient light, and after exposure to UV light.

Furthermore, the kit of the invention was found to be stable after repeated checks at different time intervals: T0 (just produced), T1 (after 7 days), T2 (after 14 days), T3 (after 21 days), T4 (after 28 days).

In summary, from the tests conducted by the present inventor it resulted that the kit according to the invention if properly stored showed a proper and sufficient stability over time, and also the different chemical-physical parameters were unaltered.

2. Clinical Trials

Clinical trials were conducted in order to examine the effectiveness of the triphasic formulation of the invention and the related targeted clinical studies.

The clinical experience lasted for 12 months of consecutive tests with subjective (perception of the treated individuals) and objective (objective clinical evaluation) evaluations on the improvement of patients' skin imperfections. In total, 32 patients were treated with the formulation as shown in the tables here below illustrated and obtained from the combination of the biphasic composition and the solid phase according to the present invention. Among all the patients treated, 30% presented not only aging problems (women aged between 40 and 75 years) but also hormonal dyschromic problems (senile or photoinduced chloasma/melasma).

The applications of the triphasic composition on the patients' skin were performed at each 21 days one from the other, and for a total of three applications.

In the studies conducted, once the triphasic formulation of the present invention was applied to the skin surface, the same was left in place on the skin and in relation to the features according to the Glougau and Fitzpatrick evaluation and the anatomical treatment area, for a period comprised between 1 minute and 5 minutes.

In the following tables it is shown the composition that gave the best expected results.

HYDROPHILIC PHASE

| | Concentration by weight |
|---|---|
| | (with respect to the total weight |
| Active ingredient | of the hydrophilic phase) |
| Deionized water | 39.8% |
| Pyruvic acid | 30% |
| Mandelic acid | 10% |
| Arginine | 7% |
| Glycolic acid 70% | 6% |
| Lactic acid 80% | 6% |
| Kojic acid | 1% |
| Azelaic acid | 0.1% |
| Salicylic acid | 0.1% |

Deionized water was added to complete the total weight of the hydrophilic phase.

LIPOPHILIC PHASE

| | Concentration by weight |
|---|---|
| | (with respect to the total weight |
| Active ingredient | of the lipophilic phase) |
| Tocopherol | 70% |
| Olive oil + | 20% |
| Bergamot essential oil | |
| Bisabolol | 10% |

SOLID PHASE IN POWDER

| | Concentration by weight |
|---|---|
| | (with respect to the total weight |
| Active ingredient | of the solid powder phase) |
| Ascorbic acid (Vitamin C) | 75% |
| Ferulic Acid | 5% |
| Hyaluronic acid | 5% |
| Sodium hyaluronate | 5% |
| Dextran-hydroxypropyltrimony | 1% |
| chloride + | |
| Palmitoyl Hexapeptide-19 | |
| Creatine | 1% |
| Arginine | 1% |
| Cysteine Hydrochloride | 1% |
| Glycine | 1% |
| Hydroxyproline | 1% |
| Lysine L monohydrochloride | 1% |
| Proline | 1% |
| Valine | 1% |
| Riboflavin (Vitamin B2) | 1% |

The clinical experience carried out in 12 months of consecutive tests has shown subjectively (perception of the treated patients) and objectively (objective clinical evaluation) not only an improvement in skin imperfections resulting from aging, such as wrinkles, skin laxity, alterations of the structure and colour of the skin, but also a positive action of the formulation of the invention already immediately after the first application on skin diseases such as acne and in its comedogenic, papular and nodular forms, chloasma/melasma, hyperpigmentation senile and photoinduced, post sclerotherapy hyperpigmentation, cutaneous xerosis and onymycosis.

In total, all 32 patients undergoing treatment with the aforementioned formulation starting from the first session showed an immediate improvement in signs of aging, while in subsequent sessions a progressive improvement in skin spots was also noted. The same behaviour was determined in acne cases (4 patients, three males and one young woman), and relating to post sclerotherapy leg hyperpigmentation, and to the only case of onychomycosis of the toe.

In all 32 cases examined there was a significant improvement in skin rejuvenation in terms of hydration, brightness, smoothness, tone, firmness and homogeneity of the complexion.

In conclusion, the clinical studies conducted by the present inventor demonstrate a synergistic action of the different active ingredients of the emulsion obtained by blending the three phases of the kit of the invention in the percentages by weight as indicated above in the tables.

With reference to the application, the present inventor used a kit according to the invention, and packaged in a vial container, wherein the hydrolipophilic composition was contained inside the vial, separately from the solid powder phase which was stored in a separate container at the vial cap. The opening of the latter through a rotary movement caused the powder to fall into the hydrolipidic composition. Through a mixing of the three phases, which was carried out by shaking the vial several times, a stable formulation was obtained, and when applied to the skin surface, it proved to be able to increase brightness, turgor, hydration, tone, complexion, counteracting the imperfections resulting from the passing time and exposure to environmental factors, respectively called chrono and photo aging.

1. A composition for the making of an emulsifiable triphasic formulation, which comprises a first liquid biphasic composition, and a second solid powder phase, wherein said first liquid biphasic composition comprises a hydrophilic phase and a lipophilic phase,
characterized in that:
said hydrophilic phase comprises at least one organic acid and at least one polar amino acid;
said lipophilic phase comprises at least one fat-soluble vitamin, at least one terpene compound and at least one essential oil; and
said solid powder phase comprises hyaluronic acid and/or a salt thereof, ferulic acid, creatine, at least one vitamin, and at least one amino acid and/or one salt thereof.

2. Composition according to claim 1, wherein said at least one organic acid in said hydrophilic phase it is selected from the group comprising pyruvic acid, mandelic acid, lactic acid, glycolic acid, kojic acid, and any combination thereof, and wherein said organic acid is preferably comprised in said hydrophilic phase in an amount ranging from 0.1% to 80% by weight with respect to the total weight of said hydrophilic phase.

3. Composition according to claim 1 or 2, wherein said hydrophilic phase further comprises azelaic acid and/or salicylic acid, and wherein said azelaic acid it is preferably comprised in said hydrophilic phase in an amount less than or equal to 10% by weight with respect to the total weight of said hydrophilic phase, and wherein said salicylic acid is preferably comprised in said hydrophilic phase in an amount less than or equal to 10% by weight with respect to the total weight of said hydrophilic phase.

4. Composition according to any of claims 1 to 3, wherein said at least one polar amino acid in said hydrophilic phase is arginine, and wherein said polar amino acid is preferably comprised in said hydrophilic phase in an amount comprised between 5% and 50% by weight with respect to the total weight of said hydrophilic phase.

5. Composition according to any of the preceding claims, wherein said hydrophilic phase comprises the following acids and amino acids in a percentage quantity by weight with respect to the total weight of said hydrophilic phase: Pyruvic acid 30%; Mandelic acid 10%; Arginine 7%; Glycolic acid (70%) 6%; Lactic acid (80%) 6%; Kojic acid 1%; 0.1% azelaic acid; Salicylic acid 0.1%; and Deionized water to complete the total weight of the hydrophilic phase.

6. Composition according to any of the preceding claims, wherein said at least one fat-soluble vitamin in said lipophilic phase is tocopherol, and wherein said fat-soluble vitamin is preferably comprised in said lipophilic phase in an amount comprised between 50% and 89% by weight with respect to the total weight of said lipophilic phase.

7. Composition according to any of the preceding claims, wherein said at least one terpene compound in said lipophilic phase is bisabolol, and wherein said terpene compound is preferably comprised in said lipophilic phase in an amount comprised between 4% and 25% by weight with respect to the total weight of said lipophilic phase.

8. Composition according to any of the preceding claims, wherein said at least one essential oil in said lipophilic phase is selected from olive oil (Olea europaea) and bergamot essential oil (Citrus bergamia), and wherein said essential oil is preferably comprised in said lipophilic phase in an amount comprised between 1% and 30% by weight with respect to the total weight of said lipophilic phase.

9. Composition according to any one of the preceding claims, wherein said lipophilic phase comprises the following active ingredients in a percentage amount by weight with respect to the total weight of said lipophilic phase: 70% tocopherol; Olive oil+Bergamot essential oil 20%; and Bisabolol 10%.

10. Composition according to any of the preceding claims, wherein said at least one vitamin in said solid powder phase is selected from ascorbic acid and riboflavin, and wherein said vitamin is preferably comprised in said solid powder phase in an amount comprised between 0.1% and 90% by weight with respect to the total weight of said solid powder phase.

11. Composition according to any of the preceding claims, wherein said at least one amino acid and/or salt thereof in said solid powder phase is selected from the group which comprises arginine, glycine, proline, hydroxyproline, valine, cysteine hydrochloride, lysine hydrochloride and any combination thereof, and wherein said amino acid and/or a salt thereof is preferably comprised in said solid powder phase in an amount comprised between 0.1% and 7% by weight with respect to the total weight of said solid powder phase.

12. Composition according to any of the preceding claims, wherein said hyaluronic acid and/or a salt thereof in said solid powder phase is comprised in an amount comprised between 5% and 10% by weight with respect to the total weight of said solid powder phase, wherein said ferulic acid is comprised in an amount comprised between 1% and 5% by weight with respect to the total weight of said solid powder phase, and wherein said creatine is comprised in an amount comprised between 0.1% and 1% by weight with respect to the total weight of said solid powder phase.

13. Composition according to any of the preceding claims, wherein said solid powder phase further comprises dextran-hydroxypropyltrimony chloride (CAS number 83855-79-2), preferably comprised in an amount less than or equal to 0.1% by weight with respect to the total weight of said solid powder phase.

14. Composition according to any of the preceding claims, wherein said solid powder phase further comprises a synthetic hexapeptide, preferably palmitoyl hexapeptide-19, and preferably comprised in said solid powder phase in an amount less than or equal to 10% by weight with respect to the total weight of said solid powder phase.

15. Composition according to any of the preceding claims, wherein said solid powder phase comprises the following active ingredients by weight with respect to the total weight of the solid powder phase:
Ascorbic acid (Vitamin C) 75%;
Ferulic acid 5%;
Hyaluronic acid 5%;
Sodium hyaluronate 5%;
Dextran-hydroxypropyltrimony chloride+Palmitoyl Hexapeptide-19 1%;
Creatine 1%;
Arginine 1%;
Cysteine Hydrochloride 1%;
Glycine 1%;
Hydroxyproline 1%;
Lysine L monohydrochloride 1%;
Proline 1%;
Valine 1%; is
Riboflavin (Vitamin B2) 1%.

16. Kit for the making of a composition of a triphasic formulation according to any of claims 1 to 15, said kit comprising:
a vial container, wherein said hydrophilic phase and said lipophilic phase of said composition is contained within said vial; and
a vial cap comprising a reservoir which comprises said solid powder phase;
the arrangement being such that following the opening of said tank in said cap, said solid powder phase is conveyed in said hydrophilic and lipophilic phases of said composition, said triphasic composition being obtained following the stirring/shaking of said vial.

17. Use of a kit for the making of a composition of a triphasic formulation according to any of claims 1 to 15, for the therapeutic treatment of a skin pathology of an individual selected from the group which includes acne pathology, alterations of melanogenesis, cutaneous xerosis, postclerotherapy hyperpigmentation and onychomycosis.

18. Use of a kit for the making of a composition of a triphasic formulation according to any of claims 1 to 15, for the cosmetic treatment of the skin of an individual, said individual not being affected by acne pathology, alterations in melanogenesis, xerosis skin, post-sclerotherapy hyperpigmentation and onychomycosis.
